(19) United States
(12) Patent Application Publication
(10) Pub. No.: US 2015/0320332 A1
(43) Pub. Date: Nov. 12, 2015

(54) SYSTEM AND METHOD FOR POTENTIATING EFFECTIVE BRAINWAVE BY CONTROLLING VOLUME OF SOUND

(71) Applicant: TIUM C&C CORPORATION, Seoul (KR)

(72) Inventors: Sanghoon LEE, Incheon (KR); Hocheol SHIN, Seoul (KR); Yeonsoon LEE, Incheon (KR); Doughyun HAN, Seoul (KR); Jaeshin KIM, Seoul (KR)

(21) Appl. No.: 14/396,153

(22) PCT Filed: Apr. 25, 2013
(86) PCT No.: PCT/KR2013/003548

§ 371 Date: Oct. 22, 2014

(30) Foreign Application Priority Data
Apr. 26, 2012 (KR) 10-2012-0043784

(51) Int. Cl. A61B 5/0482; A61B 5/04; A61B 5/0484; A61B 5/00

(52) U.S. Cl. CPC A61B 5/0482; A61B 5/742; A61B 5/0048; A61B 5/04012; A61B 5/6803; A61B 5/04842; A61B 5/04845; A61B 5/7415; A61B 5/04847

(57) ABSTRACT

Disclosed are a system and a method for potentiating effective brainwaves by controlling volume of sound. The system comprises a brainwave detector for detecting brainwaves: a sound output unit: an image output unit; and a computing means comprising: a brainwave analysis module for analyzing a brainwave signal detected by the brainwave detector; a brainwave stimulation file DB in which visual or audio information relevant to generation of effective brainwaves, or a multimedia file associated with the visual or audio information is stored; a brainwave potentiating file DB in which various audio information for potentiating the generated specific effective brainwaves is stored; and a control module designed for operating the sound output unit and the image output unit whereby if an effective brainwave is determined to be generated according to the analysis of the brainwave analysis module, a brainwave potentiating file appropriate for the effective brainwave is retrieved from the brainwave potentiating file DB, and reproduced through the sound output unit, with volume of sound controlled.

### TECHNICAL FIELD

The present invention relates to a system and a method for potentiating effective brainwaves by controlling a volume of sound. More particularly, the present invention relates to a system and a method for the potentiation of effective brainwaves utilizing a neurofeedback theory in which effective brainwaves are increased by increasing a volume of sound upon the reproduction of audio information which can induce the generation of the effective brainwaves.

### BACKGROUND ART

As society has grown more complex and competitive, there has been an increase in stress-related diseases, such as anxiety disorders, depressive disorders, headaches, sleep disturbance, etc. Although the best solution for the prevention of stress-related diseases is to avoid the source of one's stress, it is not practical or easy for modern people to do that because they are always pressed for time. When experiencing increased stress, a person is likely to undergo a stimulated condition of the sympathetic nervous system of the autonomic nervous system, that is, sympathicotonia, which may lead to the onset of a psychiatric symptom such as insomnia, depression, etc. In addition, the consequently released stress hormones may significantly reduce immunity, posing a threat to a person's health by inducing physical illnesses and even cancer.

In 1929, Hans Berger reported the concept of “brainwaves.” Brainwaves are important biological signals that indicate the activity of the human brain. Brainwaves are largely divided into five frequency bands: delta (δ) (1-4 Hz), theta (θ) (4-8 Hz), alpha (α) (8-13 Hz), beta (β) (14-30 Hz), and gamma (γ) (30-120 Hz). Most of a person's brainwaves are beta states with multiple and varying frequencies (14 Hz˜30 Hz) because they are often associated with normal waking consciousness through the five senses including talking, hearing, touching, smelling, and seeing. Alpha activity can be detected during relaxed wakefulness and increases during concentrated study, or during meditation or contemplation with closed eyes. Accounting for a border state between recognition and dreaming, theta waves are generated in a person who is slept, but not in the deepest stage of Sleep, or in a relaxed state. Theta waves are known to be associated with creativity and learning ability. Delta waves, lower in oscillation frequency than theta waves, are usually associated with the deepest stages of sleep or marked by an unconscious state. When the brain is exhibiting delta activity, growth hormones are found to be released in a vast quantity. Delta waves are also detected in patients with encephaloma, encephalitis or decreased consciousness. Gamma waves are mainly generated upon subjective consciousness and excitement.

In 1934, two scientists, E. D. Adrian and B. H. C. Matthews at the University of Cambridge, England, reported the theory of neurofeedback after finding out that, just by informing a subject of the generation of a certain wave signal, the brain automatically tends to produce the wave, as well, like the conditioned response established by Pavlov who had learned that when a bell was rung in subsequent time with food being presented to the dog in consecutive sequences, the dog will initially salivate when the food is presented, and the dog will later come to associate the ringing of the bell with the presentation of the food, and salivate upon the ringing of the bell. Now, neurofeedback has found applications in various fields, including concentration-enhancing training, intervention for ADHD and autism, and treatment for stress-related diseases such as anxiety, menstruation, insomnia, headache, depression, with a good therapeutic effect.

However, most of the neurofeedback programs developed thus far are designed to implement the output information of brainwaves in a visual form such as a video game or a graph, and thus suffer from the disadvantage of being unable to exert an effect on such brainwaves generated mostly with the eyes closed, as alpha waves.

### DISCLOSURE OF INVENTION

### Technical Problem

Accordingly, the present invention has been made keeping in mind the above problems occurring in the prior art, and an object of the present invention is to provide a system and a method for potentiating effective brainwaves utilizing a neurofeedback theory in which effective brainwaves are increased by increasing a volume of sound upon the reproduction of audio information which can induce the generation of the effective brainwaves.

### Solution to Problem

In order to achieve the above object, an aspect of the present invention is to provide a system for potentiating effective brainwaves by controlling volume of sound, comprising: a brainwave detector for detecting brainwaves: a sound output unit:

an image output unit; and a computing means comprising: a brainwave analysis module for analyzing a brainwave signal detected by the brainwave detector; a brainwave stimulation file DB in which visual or audio information relevant to generation of effective brainwaves, or a multimedia file associated with the visual or audio information is stored; a brainwave potentiating file DB in which various pieces of audio information for potentiating the generated specific effective brainwaves are stored; and a control module designed for operating the sound output unit and the image output unit whereby if an effective brainwave is determined to be generated according to the analysis of the brainwave analysis module, a brainwave potentiating file appropriate for the effective brainwave is retrieved from the brainwave potentiating file DB, and reproduced through the sound output unit, with volume of sound controlled.

In one embodiment of the present invention, the system further comprises a tactile or an olfactory stimulator wherein the control module operates the tactile or the olfactory stimulator to generate specific effective brainwaves.

In another embodiment of the present invention, the volume of sound are controlled to increase in proportion to an increase in the effective brainwaves.

In accordance with another aspect thereof, the present invention provides a method for potentiating effective brainwaves, comprising: (a) reproducing a brainwave stimulation file for generating specific effective brainwaves; (b) determining generation of effective brainwaves of interest according to the analysis of a brainwave analyzing module; and (c) reproducing an audio file for potentiating the effective brainwaves if the specific effective brainwaves are generated in step (b).

In one embodiment, the method further comprises (d) increasing a volume of sound upon the reproduction of the audio file in proportion to an increase in the level of the specific effective brainwaves.

### Advantageous Effects of Invention

The system and method for potentiating effective brainwaves by controlling volume of sound in accordance with the present invention may be useful for the suppression of an impetus causative of various addictions including alcohol addition, nicotine addiction, drug addiction, sex addiction, gambling addiction, carbohydrate addiction, horse racing addiction, and game addiction as well as the treatment of stress-related disorders such as headache, tension or anxiety, and slight depression because the system can induce the brain to increase alpha waves that result in psychological stability, thereby reducing the tension and nervousness which form an impetus to indulge in addiction.

In addition to the psychiatric treatment, the system and method of the present invention can be used as a means for effectively transmitting emotional expressions or messages to general persons or persons under a specific condition. For example, when a voice file of a loving or hopeful message is geared with alpha waves, the system and method of the present invention can be used to increase an amiable feeling between lovers or to encourage a failure.

### BRIEF DESCRIPTION OF DRAWINGS

FIG. 1 is a schematic view illustrating a setup of a system for potentiating effective brainwaves by controlling volume of sound in accordance with an embodiment of the present invention

FIG. 2 is a functional block diagram of the system shown in FIG. 1.

FIG. 3 is a flow chart explaining a method for potentiating effective brainwaves by controlling a volume of sound in accordance with the present invention.

FIG. 4 is a schematic view showing the appearance of a system for potentiating effective brainwaves by controlling a volume of sound in accordance with another embodiment of the present invention.

FIG. 5 is a functional block diagram of the system shown in FIG. 4.

### DESCRIPTION OF REFERENCE NUMERALS

10: Image output unit,

20: Sound output unit,

30: Tactile stimulator,

40: Olfactory stimulator,

50: Brainwave detector,

60: Keyboard,

70: Mouse,

80: Output interface,

90: Input interface,

100: Computer,

110: Microprocessor,

120: Memory controller,

125: Graphic controller,

130: I/O controller,

140: Network controller,

150: Main memory device,

160: Hard disc,

161: Operating system,

162, 310: Control module,

163, 360: Brainwave analysis module,

164, 340: Brain stimulation file DB,

165, 350: Brainwave potentiating file DB

### MODE FOR THE INVENTION

Reference now should be made to the drawings, in which the same reference numerals are used throughout the different drawings to designate the same or similar components, to describe preferred embodiments of the system for potentiating effective brainwaves by controlling volume of sound in accordance with the present invention.

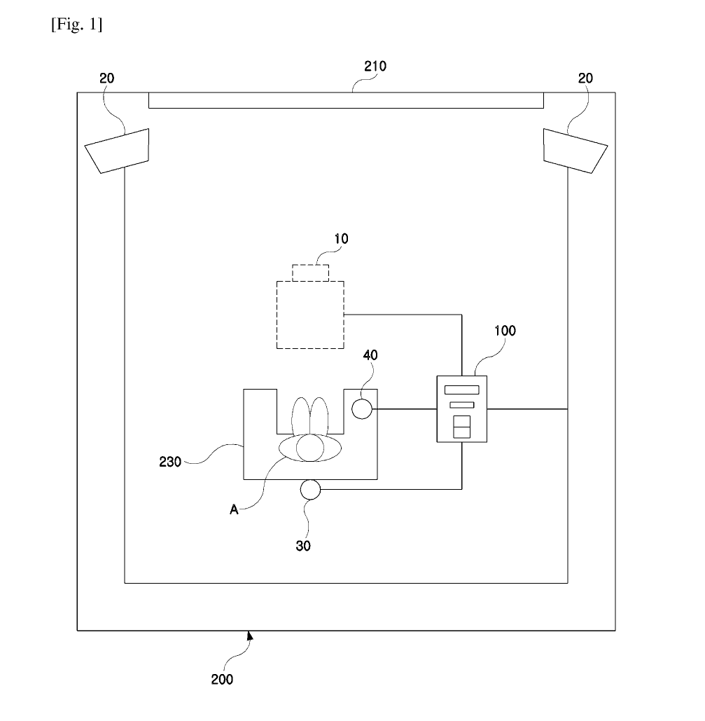

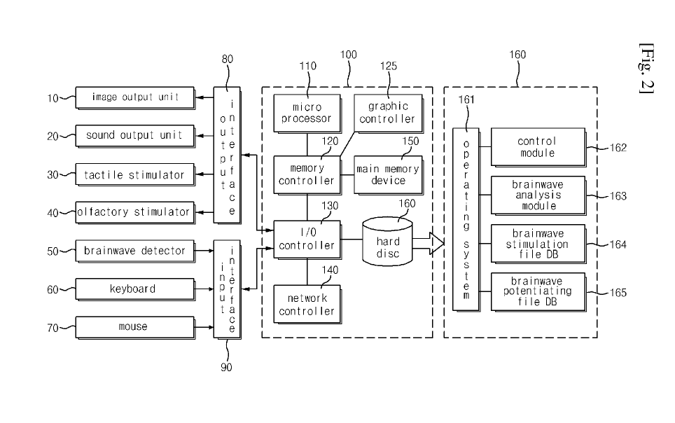

With reference to FIG. 1, there is a setup of a system for potentiating effective brainwaves by controlling volume of sound in accordance with an embodiment of the present invention while FIG. 2 is a functional block diagram of the system shown in FIG. 1. As can be seen in FIGS. 1 and 2, the system for potentiating effective brainwaves by controlling volume of sound in accordance with an embodiment of the present invention can be set in a space, such as a physical therapy department 200 of a hospital or a rehabilitation facility. The system may be generally divided into a computer 100 installed with an effective brainwave potentiating program, an output module comprising an image output unit 10 and a sound output unit 20 for outputting various images and sounds to effect muscle relaxation and the generation of specific effective brainwaves, respectively, and a tactile stimulator 30 and an olfactory stimulator 40 for generating the sense of touch and the sense of smell in conjunction with muscle relaxation or the generation of specific effective brainwaves, respectively, an output interface 80 for interfacing the output module with the computer 100, an input module comprising a keyboard 60 and a mouse 70, both for inputting or setting information relevant to the potentiation of effective brainwaves, and a brainwave detector 50, applied to or worn by a patient A, for determining the extent of muscle relaxation or whether the specific effective brainwaves are generated, and an input interface 90 for interfacing the input module with the computer 100.

In the constitution of the system, the sound output 20 may be typically implemented with a speaker. For example, a multichannel speaker system capable of generating stereo sounds, e.g., a 5.1 channel speaker system, may be employed to maximize sound effects. The image output unit 10 may be simply a computer monitor, or may be preferably embodied by a beam projector adapted to project 2D or 3D images on a screen 210 installed in front of the patient in the physical therapy department 200, or by an HMD (head mounted display).

The tactile stimulator 30 is designed to produce visual or auditory stimuli useful for the generation of specific effective brainwaves. For example, the tactile stimulator may be an ultrasound generator for producing ultrasound that is harmless to the body, or a vibrator. In this regard, the vibrator may be set up within the seat or back of a chair 230 for the patient. The ultrasound generator may be placed at a suitable place in the physical therapy department 200. The olfactory stimulator 40 may be generally used to induce muscular or metal relaxation, thereby generating specific effective brainwaves, for example, alpha waves. In one embodiment, the olfactory stimulator 40 may be a scent generator. The olfactory stimulator 40 may comprise a heater for igniting a solid or liquid scent source, and a blast fan for forcibly dispersing the generated scent in air. In another embodiment, the olfactory stimulator 40 may be composed of an independent natural or artificial scent source. The brainwave detector 50 may comprise a typical brainwave detection sensor. The output interface 80 and the input interface 90 may be embodied by, for example, respective USB interfaces (hubs).

Turning to the computer 100, it may be a general-purpose personal computer, the hardware constitutional elements of which include a microprocessor 110 as a central process unit (CPU), a main memory unit 150 composed of a RAM (random access memory), a memory controller 120 for controlling the main memory unit 150, a graphic controller 125 composed of a graphic memory for processing various graphics, an I/O controller 130 for controlling the input/output of data through the input interface 90 and the output interface 80, a network controller 140, such as a LAN card, for supporting telecommunication such as Internet communication, and a hard disc 160 installed with the effective brainwave-potentiating program of the present invention.

In the computer system, the hard disc 160 may store the effective brainwave-potentiating program that can be operated on the basis of an operating system 161, such as the Windows XP series by Microsoft, and a database in which various relevant data and files are systemically managed. The database may comprise a brainwave stimulation file DB 164 in which images or sounds relevant to the generation of effective brainwaves, or a multimedia file associated with the images or sounds are stored, and a brainwave potentiating file DB 165 in which various sounds for potentiating the generated specific effective brainwaves are stored.

Next, the effective brainwave-potentiating program may comprise a brainwave analysis module 163 for analyzing the brainwave signal detected by the brainwave detector 50 to determine what the brainwave being generated is, and a control module 162 which is designed to operate the tactile stimulator 30 or the olfactory stimulator 40 to generate the effective brainwaves or to retrieve a suitable brainwave stimulation file from the brainwave stimulation file DB to output a brainwave stimulus through the sound output unit 20 or the image output unit 10, whereby if an effective brainwave is determined to be generated according to the analysis of the brainwave analysis module 163, a brainwave potentiating file proper to the effective brainwave is retrieved from the brainwave potentiating file DB 165 and outputted through the sound output unit 20, with the volume of sound controlled with time.

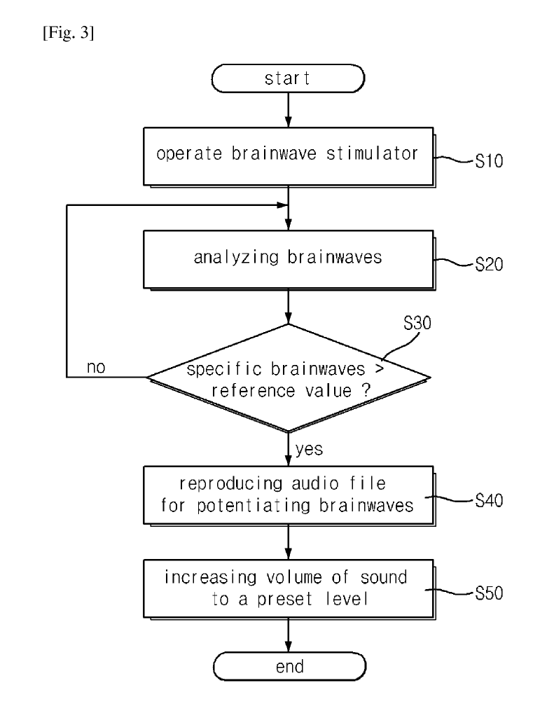

FIG. 3 is a flow chart which explains, on the basis of the control module 162, a method for potentiating effective brainwaves by controlling a volume of sound. In the method for potentiating effective brainwaves using a change in volume of sound according to the present invention, as shown in FIG. 3, a stimulator for endowing psychological stability, such as an image output unit 10, a sound output unit 20, a tactile stimulator 30, or an olfactory stimulator 40, is operated to generate alpha waves (S10). In this regard, a brainwave stimulation file, for example, an image of a natural landscape, such as a peaceful sea or prairie scenery (hereinafter referred to as “background scenery”), or a piece of tranquil music (hereinafter referred to as “background music”), which is stored in the brainwave stimulation file DB 164, is outputted through the image output unit 10 and the sound output unit 20.

If the psychological state is stabilized with time in this condition, alpha waves increase among the neural oscillations originating from the occipital lobe. In step S20, the brainwaves detected in the brainwave detector 50 are analyzed by the brainwave analysis module 163, and according to the analysis result, the generation of specific effective brainwaves is determined by examining whether the amplitude of specific effective brainwaves, for example, alpha waves exceeds a reference value, in step S30.

When the specific effective brainwaves, for example, alpha waves are produced at a level below the reference value in step S30, step S20 is repeated. In contrast, when their amplitude is over the reference value, a corresponding brainwave potentiating file is retrieved from the brainwave potentiating file DB 165 and outputted through the sound output unit 40. If the specific effective brainwaves are alpha waves, the brainwave potentiating file may contain sound information, such as a tranquil ripple or wind sound. At first, the file is reproduced at a lowest volume, which acts as a default value. When the amplitude of the alpha waves is lower than the reference value, only the background image or sound is outputted. As soon as the amplitude exceeds the reference value, a brainwave potentiating sound is reproduced with the background music remaining unchanged in volume.

In step S50, the volume of the brainwave potentiating sound is gradually increased to a predetermined level to potentiate the specific effective brainwaves, for example, alpha waves. In this context, the volume of sound may be increased either by a predetermined level at regular time intervals or in proportion to the increment of the amplitude of alpha waves per time. For example, as mentioned above, a patient looks at marine scenery without a ripple sound, during which when alpha waves are generated and increase to a reference value, a ripple sound is outputted. Subsequently, the volume of the ripple sound is increased as alpha waves increase, with eyes closed or open, whereby the amplitude of alpha waves can be potentiated on the basis of the neurofeedback theory.

In addition to natural sounds, music, animal sounds or a human voice, like a lover's voice saying, “I love you,” may be used as a brainwave potentiating sound.

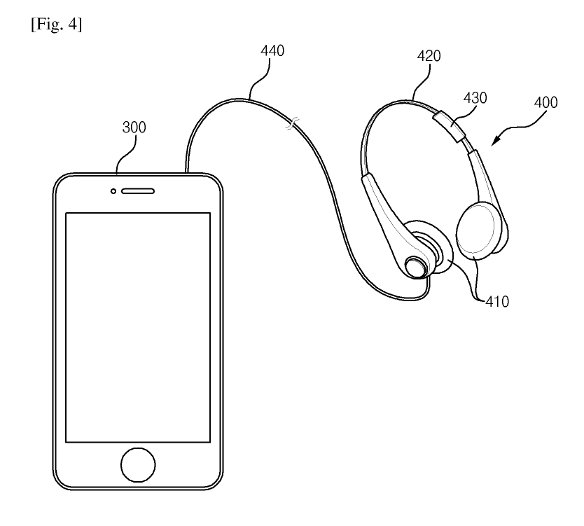

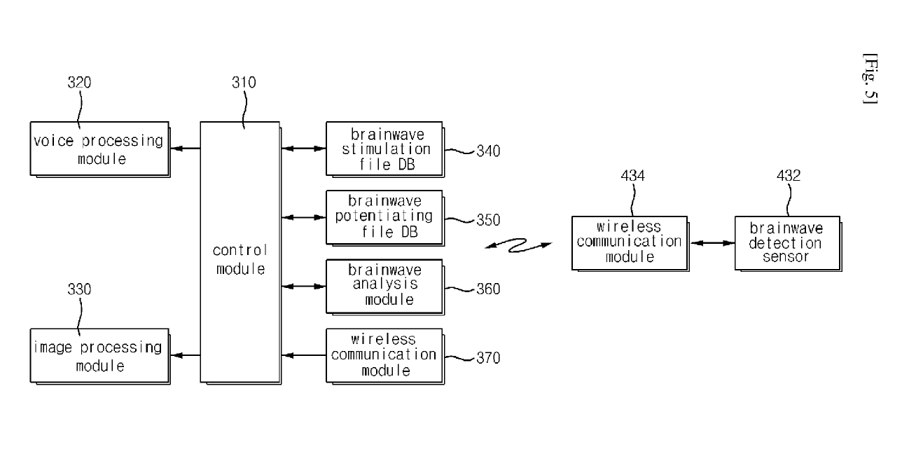

FIG. 4 is a schematic view showing the appearance of a system for potentiating effective brainwaves by controlling a volume of sound in accordance with another embodiment of the present invention. FIG. 5 is a functional block diagram of the system shown in FIG. 4. FIGS. 4 and 5 illustrate an application of the system and method for potentiating effective brainwaves by controlling volume of sound in accordance with the present invention to a personal mobile device 300 such as a smart phone or a smart pad. In this case, a brainwave detector 430 is set on a hair band 420 of a headset connected through a cable 400 to the personal mobile device 300.

As shown in FIGS. 4 and 5, the system for potentiating effective brainwaves by controlling volume of sound according to another embodiment of the present invention may be installed as an application in a personal mobile device. In the figures, reference numerals 310, 340, 350, and 360 respectively represent a control module, a brainwave stimulation file DB, a brainwave potentiating file DB, and a brainwave analysis module which are constitutionally the same as explained in FIG. 2, and reference numerals 320 and 330 are a sound processing module and an image processing module, respectively.

Meanwhile, the brainwave detector 430 may comprise a brainwave detection sensor 432 and a wireless communication module 434 for transmitting detected brainwaves to the personal mobile device 300. For the wireless communication module, a Bluetooth module generally employed in personal mobile devices may be adopted. In the figure, reference numeral 370 represents a wireless communication module applied to the personal mobile device 300.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A system for potentiating effective brainwaves by controlling volume of sound, comprising:
a brainwave detector for detecting brainwaves:
a sound output unit:
an image output unit; and
a computing means comprising:
a brainwave analysis module for analyzing a brainwave signal detected by the brainwave detector;
a brainwave stimulation file DB in which visual or audio information relevant to generation of effective brainwaves, or a multimedia file associated with the visual or audio information is stored;
a brainwave potentiating file DB in which various audio information for potentiating the generated specific effective brainwaves is stored; and
a control module designed for operating the sound output unit and the image output unit whereby if an effective brainwave is determined to be generated according to the analysis of the brainwave analysis module, a brainwave potentiating file proper to the effective brainwave is retrieved from the brainwave potentiating file DB, and reproduced through the sound output unit, with volume of sound controlled.

2. The system of claim 1, wherein the system further comprises a tactile stimulator, and the control module operates the tactile stimulator to generate a specific effective brainwave.

3. The system of claim 2, wherein the system further comprises an olfactory stimulator, and the control module operates the olfactory stimulator to generate a specific effective brainwave.

4. The system of claim 1, wherein the control module is designed to increase the volume of sound in proportion to an increase in the specific effective brainwave.

5. A system for potentiating effective brainwaves by controlling volume of sound, comprising:
a sound processing module for outputting a sound:
an image processing module for outputting an image;
a brainwave analysis module for receiving a brainwave through wireless communication and analyzing the brainwave, said brainwave being detected by a brainwave detector for detecting brainwaves;
a brainwave stimulation file DB in which visual or audio information relevant to generation of effective brainwaves, or a multimedia file associated with the visual or audio information is stored;
a brainwave potentiating file DB in which various audio information for potentiating the generated specific effective brainwaves is stored; and
a control module designed for operating the sound output unit and the image output unit whereby if an effective brainwave is determined to be generated according to the analysis of the brainwave analysis module, a brainwave potentiating file appropriate for the effective brainwave is retrieved from the brainwave potentiating file DB, and reproduced through the sound output unit, with volume of sound controlled.

6. The system of claim 5, being applied to a personal mobile device selected from the group consisting of a smart phone and a smart pad.

7. The system of claim 6, further comprising a headset for the smart phone or the smart pad, wherein the headset comprises a brainwave detector which is set in a hair band of the headset and comprises a brainwave detection sensor for detecting brainwaves, and a wireless communication module for transmitting detected brainwaves to the smart phone or the smart pad.

8. The system of claim 5, wherein the control module is designed to increase the volume of sound in proportion to an increase in the specific effective brainwave.

9. A method for potentiating effective brainwaves, comprising:
(a) reproducing a brainwave stimulation file for generating specific effective brainwaves;
(b) determining generation of effective brainwaves of interest according to the analysis of a brainwave analyzing module; and
(c) reproducing an audio file for potentiating the effective brainwaves if the specific effective brainwaves are generated in step (b).

10. The method of claim 9, further comprising:
(d) increasing a volume of sound upon the reproduction of the audio file in proportion to an increase in the level of the specific effective brainwaves.
